(19) United States
(12) Patent Application Publication
(10) Pub. No.: US 2016/0310094 A1
(43) Pub. Date: Oct. 27, 2016

(54) MEDICAL IMAGE PROCESSING APPARATUS

(71) Applicants: National Cancer Center, Chuo-ku (JP); Toshiba Medical Systems Corporation, Otawara-shi (JP)

(72) Inventors: Tatsushi KOBAYASHI, Kashiwa (JP); Shinsuke TSUKAGOSHI, Nasushiobara (JP)

(73) Assignees: National Cancer Center, Chuo-ku (JP); Toshiba Medical Systems Corporation, Otawara-shi (JP)

(21) Appl. No.: 15/138,665

(22) Filed: Apr. 26, 2016

(30) Foreign Application Priority Data
Apr. 27, 2015 (JP) 2015-090341
Apr. 22, 2016 (JP) 2016-086428

(51) Int. Cl. A61B 6/00; G06T 7/00; A61B 8/08; A61B 5/00; A61B 6/03; A61B 5/055

(52) U.S. Cl. CPC A61B 6/5217; A61B 6/032; A61B 5/055; A61B 8/0891; A61B 6/5223; A61B 6/463; A61B 6/504; A61B 5/7282; G06T 7/0012; G06T 2207/10081; G06T 2207/10088; G06T 2207/30096

(57) ABSTRACT

A medical image processing apparatus according to one embodiment includes image processing circuitry. The image processing circuitry set a measurement target slice intersecting with a tubular tissue region of a tubular tissue with likelihood of tumor infiltration with respect to a plurality of three-dimensional images in a plurality of phases. The image processing circuitry measure the morphological index value of the measurement target slice of the tubular tissue region over a plurality of phases. The image processing circuitry determine, based on a change in morphological index value over a plurality of phases, whether a tumor has infiltrated the tubular tissue.

### CROSS-REFERENCE TO RELATED APPLICATIONS

This application is based upon and claims the benefit of priority from the prior Japanese Patent Application No. 2015-090341, filed Apr. 27, 2015 and the prior Japanese Patent Application No. 2016-086428, filed Apr. 22, 2016, the entire contents of all of which are incorporated herein by reference.

### FIELD

Embodiments described herein relate generally to a medical image processing apparatus.

### BACKGROUND

Other organ infiltration diagnosis concerning advanced esophageal cancer is an important factor associated with a treatment method or prognosis based on the presence/absence of infiltration (literature 1 (Ando N, Kato H, Igaki H, et al., “A Randomized Trial Comparing Postoperative Adjuvant Chemotherapy with Cisplatin and 5-Fluorouracil Versus Preoperative Chemotherapy for Localized Advanced SquamousCell Carcinoma of the Thoracic Esophagus (JCOG9907)”, Ann Surg Oncol, January 2012, vol. 19(1), pp. 68-74), and literature 2 (Ishida K, Ando N, Yamamoto S, et al., “Phase II study of cisplatin and 5-fluorouracil with concurrent radiotherapy in advanced squamous cell carcinoma of the esophagus: a Japan Esophageal Oncology Group (JEOG)/Japan Clinical OncologyGroup trial (JCOG9516)”, Jpn J Clin Oncol, October 2004, vol. 34(10), pp. 615-619”). Image evaluation is performed by using a CT image obtained at a deep breath. When no other organ infiltration is recognized, stage T3 is determined, whereas when other organ infiltration is recognized, stage T4 is determined, thereby determining a disease stage before surgery (staging). It is reported that an existing method exhibits an accuracy of 90% to 98%, sensitivity of 75% to 100%, and a specificity of 88% to 100% (see literature 3 (Thompson W M, Halvorsen R A, Foster W L Jr, et al., “Computed tomography for staging esophageal and gastroesophageal cancer: reevaluation”, AJR Am J Roentgenol, November 1983, vol. 141(5), pp. 951-958)). In this report, this method exhibits an accuracy of 93%, a sensitivity of 97%, and a specificity of 88% concerning infiltration into the trachea and bronchi. In actual clinical practices, however, it is sometimes difficult to perform infiltration evaluation for the purpose of determining stage T3 or T4 by using an existing method. For this reason, there have been no few cases which have been difficult to diagnose before surgery.

### BRIEF DESCRIPTION OF THE DRAWING

FIG. 1 is a block diagram showing the arrangement of a medical image processing apparatus according to the first embodiment;

FIG. 2 is a view schematically showing the positional relationship between the esophagus, the trachea, and the bronchi;

FIG. 3 is a flowchart showing a procedure for infiltration determination processing to be performed under the control of control circuitry in FIG. 1;

FIG. 4 is a view showing an example of a bronchial region extracted by a slice setting function in step SA1 in FIG. 3;

FIG. 5 is a view showing an example of a slice image of a measurement target slice set in step SA1 in FIG. 3;

FIG. 6 is a view showing an example of setting a measurement region on a slice image of a measurement target slice set in step SA1 in FIG. 3;

FIG. 7 is a view schematically showing a temporal change in the form of a bronchial region in a measurement target slice at each of T3 and T4 concerning step SA3 in FIG. 3;

FIG. 8 is a graph showing a temporal change in luminal area as one of morphological index values at each of T3 and T4 concerning step SA3 in FIG. 3;

FIG. 9 is a graph showing a temporal change in luminal area as one of morphological index values at T4 and in each of a measurement region and a normal region concerning step SA3 in FIG. 3;

FIG. 10 is a view showing a display example of a determination result displayed by display circuitry concerning step SA4 in FIG. 3;

FIG. 11 is a block diagram showing the arrangement of a medical image processing apparatus according to the second embodiment;

FIG. 12 is a flowchart showing a procedure for infiltration determination processing to be performed under the control of control circuitry according to the second embodiment;

FIG. 13 is a view schematically showing a procedure for infiltration determination processing according to the second embodiment;

FIG. 14 is a flowchart showing a procedure for infiltration determination to be performed under the control of control circuitry according to the third embodiment; and

FIG. 15 is a view schematically showing a procedure for infiltration determination processing according to the third embodiment.

### DETAILED DESCRIPTION

In general, according to one embodiment, a medical image processing apparatus includes image processing circuitry which processes a plurality of three-dimensional images in a plurality of phases. The image processing circuitry set a slice intersecting with a tubular tissue region of a tubular tissue with likelihood of tumor infiltration with respect to the plurality of three-dimensional images in the plurality of phases. The image processing circuitry measure morphological index values on the slice of the tubular tissue region over the plurality of phases. Based on a change in morphological index value over the plurality of phases, the image processing circuitry determine whether the tumor has infiltrated the tubular tissue.

The medical image processing apparatus according to this embodiment will be described below with reference to the accompanying drawing.

### First Embodiment

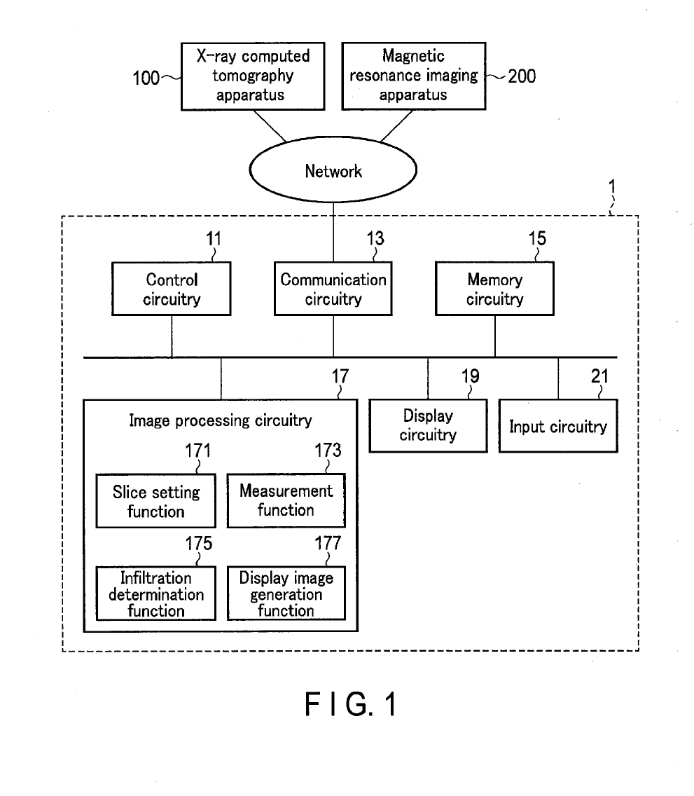

FIG. 1 is a block diagram showing the arrangement of a medical image processing apparatus 1 according to the first embodiment. As shown in FIG. 1, the medical image processing apparatus 1 is a computer apparatus which processes a medical image generated by a medical modality. For example, the medical image processing apparatus 1 is communicably connected to a medical modality via a network. Medical modalities according to this embodiment include, for example, an X-ray computed tomography apparatus 100 and a magnetic resonance imaging apparatus 200. The medical modalities according to the embodiment are not limited to the X-ray computed tomography apparatus 100 and the magnetic resonance imaging apparatus 200, and may include, for example, any types of medical modalities such as an ultrasonic diagnostic apparatus and a nuclear medicine diagnostic apparatus.

Note that the medical image processing apparatus 1 according to this embodiment need not always be connected to medical modalities and medical image archiving apparatuses. In addition, the medical image processing apparatus may be a computer as a component of a medical modality.

As shown in FIG. 1, the medical image processing apparatus 1 according to the first embodiment includes control circuitry 11 as a main unit, communication circuitry 13, memory circuitry 15, image processing circuitry 17, display circuitry 19, and input circuitry 21.

The communication circuitry 13 are a communication interface for performing data communication with other apparatuses via a network. The communication circuitry 13 communicate with medical modalities such as the X-ray computed tomography apparatus 100 and the magnetic resonance imaging apparatus 200 or medical image archiving apparatuses such as a PACS (Picture Archiving and Communication System) (not shown) via a network. For example, the communication circuitry 13 receive a plurality of three-dimensional images (volume data) in a plurality of phases, so-called four-dimensional image data, from an X-ray computed tomography apparatus or magnetic resonance imaging apparatus. Alternatively, the communication circuitry 13 may receive a plurality of three-dimensional images in a plurality of phases from a medical image archiving apparatus or the like (not shown).

Memory circuitry 15 are a storage device such as an HDD (Hard Disk Drive), SSD (Solid State Drive), or integrated circuit storage device which stores various types of information. In addition, the memory circuitry 15 may include a driver or the like which reads and writes various types of information from and on a portable storage medium. The memory circuitry 15 store a plurality of three-dimensional images in a plurality of phases. The plurality of three-dimensional images are acquired by imaging the esophagus of an object over a plurality of phases using a medical modality. In addition, the memory circuitry 15 store an image processing program and the like associated with infiltration determination processing (to be described later).

The image processing circuitry 17 include, as hardware resources, an arithmetic device (processor) such as a CPU (Central Processing Unit), MPU (Micro Processing Unit), or GPU (Graphics Processing Unit) and storage devices (memories) such as a ROM (Read Only Memory) and a RAM (Random Access Memory). The image processing circuitry 17 processing three-dimensional images in a plurality of phases and determines whether a tumor has infiltrated a tubular tissue around the tissue where the tumor has occurred. The processing of determining the presence/absence of tumor infiltration into the tubular tissue, which is performed by the image processing circuitry 17 will be referred to as infiltration determination processing. The image processing circuitry 17 implement a slice setting function 171, a measurement function 173, an infiltration determination function 175, and a display image generation function 177 by executing the above image processing programs.

The image processing circuitry 17 execute the slice setting function 171 to set a slice (to be referred to as a set slice hereinafter) intersecting with an image region concerning a tubular tissue with likelihood of tumor infiltration (to be referred to as a tubular tissue region hereinafter) with respect to a plurality of three-dimensional images in a plurality of phases. Tubular tissues with likelihood of tumor infiltration include, for example, the trachea, bronchi, and blood vessels.

The image processing circuitry 17 execute the measurement function 173 to measure a morphological index value on a set slice of a tubular tissue region over a plurality of phases. A morphological index value is an index value concerning a geometrical feature of a tubular tissue region.

The image processing circuitry 17 execute the infiltration determination function 175 to determine, based on a change in morphological index value over a plurality of phases, whether a tumor has infiltrated the tubular tissue. The display circuitry 19 display the determination result.

The image processing circuitry 17 execute the display image generation function 177 to generate a two-dimensional display image by performing three-dimensional image processing for each of a plurality of three-dimensional images. The display image generation function 177 generates a display image by performing three-dimensional image processing such as volume rendering, surface volume rendering, image value projection processing, MPR (Multi-Planar Reconstruction) processing, or CPR (Curved MPR) processing for a three-dimensional image.

The display circuitry 19 display, on a display device, various types of information, e.g., a determination result obtained by the infiltration determination function 175 and a display image generated by the display image generation function 177. It is possible to properly use, as a display device, for example, a CRT display, liquid crystal display, organic EL display, LED display, plasma display, or another arbitrary display known in this technical field.

The input circuitry 21 accept various types of commands or information inputs from the user using an input device. As an input device, a keyboard, mouse, various types of switches, and the like can be used.

The control circuitry 11 include, as hardware resources, an arithmetic device such as a CPU or MPU and storage devices such as a ROM and a RAM. The control circuitry 11 function as the main unit of the medical image processing apparatus 1 according to this embodiment. For example, the control circuitry 11 read out an image processing program stored in the memory circuitry 15 and controls each unit in the medical image processing apparatus 1 in accordance with the readout image processing program.

Infiltration determination processing performed under the control of the control circuitry 11 according to this embodiment will be described in detail below by exemplifying other organ diagnosis concerning esophagus cancer as a clinical application example.

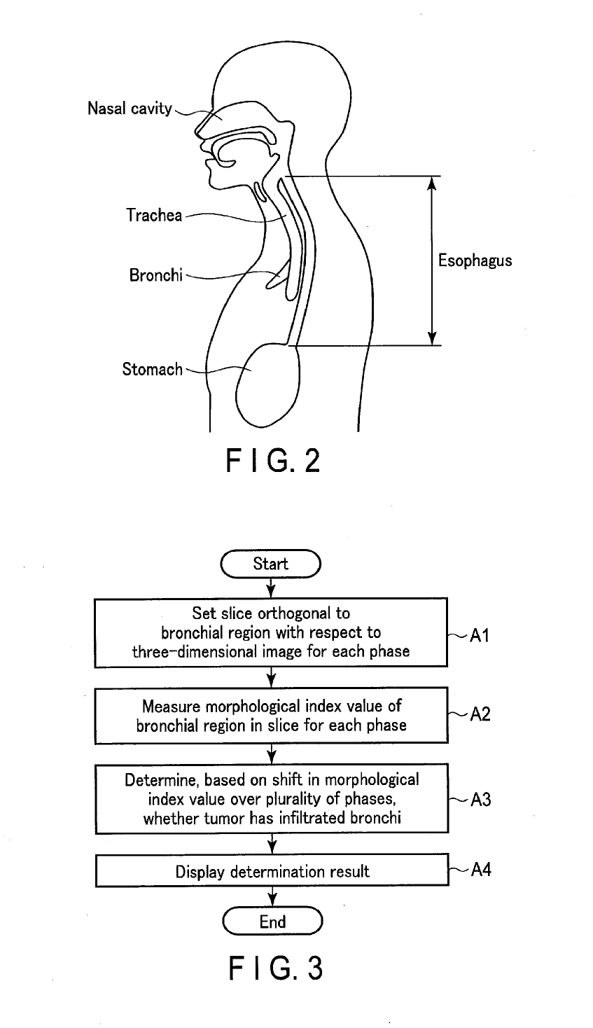

FIG. 2 is a view schematically showing the positional relationship between the esophagus, trachea, and bronchi. As shown in FIG. 2, the esophagus is a tubular tissue that connects the throat to the stomach. The trachea is located on the front surface side of the esophagus in the upper part of the chest. The trachea and the esophagus separate from each other in the neck. The trachea branches into the bronchi, more specifically, the two main bronchi, at the bifurcation of the trachea. Each main bronchi further branches into a plurality of bronchioles and into a plurality of alveolar bronchioles toward the periphery.

The esophageal wall can be divided, from the inside to the outside, into the four layers, namely the mucosal layer, the submucosal layer, the proper muscular layer, and the adventitia. The stage of esophagus cancer is represented by a T factor as a wall invasion depth. At stage T1a, cancer remains in the mucosal layer. At stage T1b, cancer remains in the submucosal layer. At stage T2, cancer remains in the proper muscular layer. At stage T3, cancer has infiltrated the adventitia. At stage T4, cancer has infiltrated a peripheral tissue of the esophagus. The esophagus cancer can infiltrate a peripheral tissue such as a tubular tissue such as the trachea, bronchi, and blood vessels.

Clinically, it is thought that cancer up to stage T3 can be surgically removed with a high probability, and cancer which has advanced to stage T4 is difficult to surgically remove. When it is diagnosed before surgery that the cancer has advanced to stage T4, surgery is sometimes avoided. That is, it should be avoided to diagnose that cancer is at stage T4 in spite of the fact that the cancer is actually at stage T3.

The medical image processing apparatus 1 according to the first embodiment processes a plurality of three-dimensional images in a plurality of phases and determines whether the esophagus cancer is at stage T4, that is, esophagus cancer has infiltrated a tubular tissue with likelihood of tumor infiltration.

FIG. 3 is a flowchart showing a procedure for infiltration determination processing to be performed under the control of the control circuitry 11 according to the first embodiment. Assume that a medical modality such as an X-ray computed tomography apparatus or magnetic resonance imaging apparatus has acquired a plurality of three-dimensional images in a plurality of phases by imaging an imaging region including the esophagus of an object before step S1. For example, the X-ray computed tomography apparatus scans the object with X-rays by using a gantry including an X-ray tube and an X-ray detector. The X-ray detector outputs raw data concerning a plurality of views. A noncontact data transmitter transmits the data to a reconstruction apparatus. The reconstruction apparatus reconstructs a plurality of three-dimensional images in a plurality of phases based on the raw data concerning a plurality of views transmitted from the gantry. Note that the object repeatedly breathes during an imaging period. This makes it possible to capture the slight extension of the membranous portion of the trachea accompanying the respiration of the object in the three-dimensional images in the plurality of phases. The plurality of acquired three-dimensional images in the plurality of phases are transmitted to the medical image processing apparatus 1 via a network and stored in the memory circuitry 15.

Upon reception of an instruction to start infiltration determination processing, the control circuitry 11 read out a plurality of three-dimensional images in a plurality of phases from the memory circuitry 15 and supplies them to the image processing circuitry 17. As shown in FIG. 3, the control circuitry 11 cause the image processing circuitry 17 to execute the slice setting function 171 (step SA1). In step SA1, the image processing circuitry 17 set a slice intersecting with a tubular tissue region of a tubular tissue with respect to which the likelihood of esophagus cancer infiltration should be determined with respect to a plurality of three-dimensional images in a plurality of phases. A set slice will be referred to as a measurement target slice hereinafter. For the sake of concreteness, assume that a tubular tissue with likelihood of esophagus cancer infiltration is the bronchi.

The user may designate a measurement target slice intersecting with an image region of the bronchi (to be referred to as a bronchial region hereinafter) via the input circuitry 21 or a measurement target slice may be automatically set by image processing.

A case in which the user performs designation via the input circuitry 21 will be described. The image processing circuitry 17 execute the display image generation function 177 to generate a slice image of the slice automatically set by the user via the input circuitry 21 based on a three-dimensional image. The display circuitry 19 display the generated slice image. For example, when imaging the left main bronchi, the image processing circuitry 17 can obtain a double oblique image orthogonal to the bronchi by performing slice conversion of a three-dimensional image at an oblique position twice. The user observes a slice image while changing the slice via the input circuitry 21, and designates a slice that allows diagnosis of the infiltration of esophagus cancer into the bronchi. The image processing circuitry 17 execute the slice setting function 171 to set the designated slice as a measurement target slice.

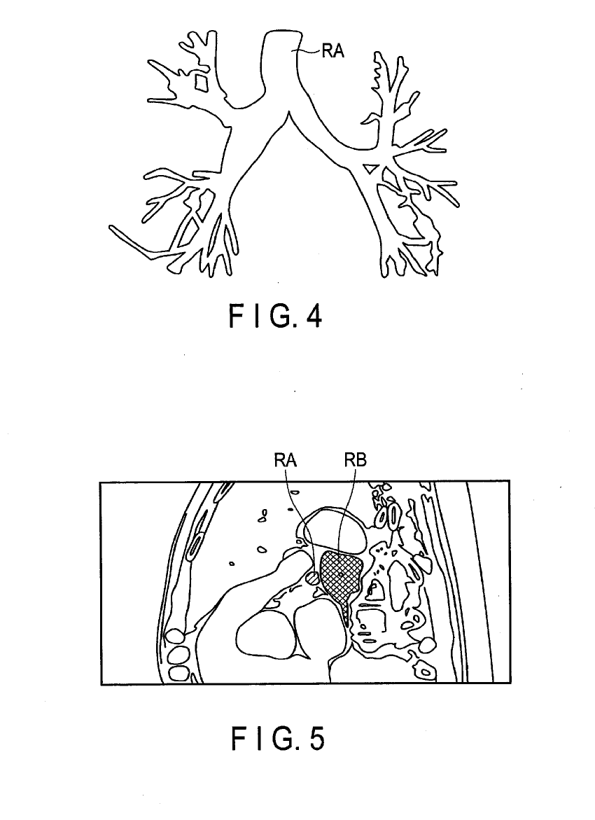

A method of automatically setting a slice by image processing will be described. First of all, as shown in FIG. 4, the image processing circuitry 17 execute the slice setting function 171 to extract a bronchial region (more specifically, an image region of a lumen (to be referred to as a luminal region hereinafter)) RA included in a three-dimensional image by a region expansion method, a template matching method, or existing image processing such as threshold processing. The image processing circuitry 17 then set a measurement target slice based on the local shape of the bronchial region RA. More specifically, the image processing circuitry 17 set a plurality of orthogonal slices along the central axis of the bronchial region RA with respect to a three-dimensional image, and measures the shape of the bronchial region RA on each set orthogonal slice. On each orthogonal slice, the bronchial region RA having no esophagus cancer around it has an almost elliptic shape. As the esophagus cancer advances, the bronchial region RA exhibits a distorted crescent shape. That is, the image processing circuitry 17 preferably set an orthogonal slice with the bronchial region RA having a crescent shape as a measurement target slice.

As another image processing, the image processing circuitry 17 specify an image region (to be referred to as an esophagus cancer region hereinafter) associated with the esophagus cancer included in a three-dimensional image in accordance with an input operation by the user via the input circuitry 21 or existing image processing. The image processing circuitry 17 then preferably specify a bronchial region existing around the specified esophagus cancer region and sets a slice orthogonal to the central axis of the specified bronchial region as a measurement target slice.

The image processing circuitry 17 may execute the slice setting function 171 to set a measurement target slice in each phase by the above method, set a measurement target slice in one phase by the above method, or track the set measurement target slice in another phase by image processing such as a tracking method to set a measurement target slice.

When a measurement target slice is set by the above method, the image processing circuitry 17 execute the display image generation function 177. The image processing circuitry 17 execute the display image generation function 177 to generate a slice image of the measurement target slice based on a three-dimensional image for each phase. FIG. 5 is a view showing an example of a slice image of the measurement target slice set in step SA1. As shown in FIG. 5, the measurement target slice is orthogonal to the central axis of the bronchial region RA, and the bronchial region RA and the esophagus cancer region RB are depicted in a slice image of the measurement target slice.

Upon execution of step SA1, the control circuitry 11 cause the image processing circuitry 17 to execute the measurement function 173 (step SA2). In step SA2, the image processing circuitry 17 measure a morphological index value on a slice of the bronchial region over a plurality of phases. The measurement of a morphological index value will be described below. First of all, the image processing circuitry 17 set a region of interest (to be referred to as a measurement region hereinafter) for the measurement of a morphological index value of the bronchial region on the measurement target slice.

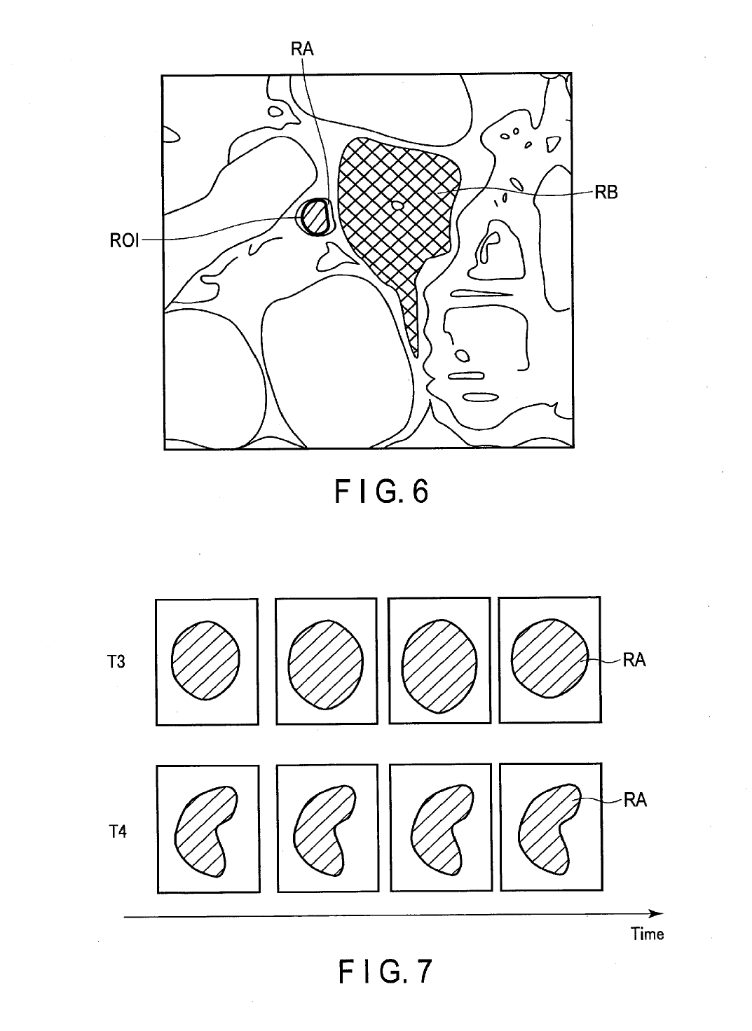

FIG. 6 is a view showing an example of setting a measurement region ROI on a slice image of the measurement target slice set in step SA1. As shown in FIG. 6, the image processing circuitry 17 set the measurement region ROI in the bronchial region, more specifically, the luminal region RA in accordance with an instruction issued by the user via the input circuitry 21 or automatically by image processing. More specifically, the measurement region ROI may be set so as to be inscribed in the luminal region RA of the bronchial region or may be set so as to circumscribe the luminal region RA. The image processing circuitry 17 may set the measurement region ROI in each phase by the above method or may set the measurement region ROI in a given phase by the above method while setting the measurement region ROI in another phase by tracking the set measurement region ROI by image processing such as a tracking method.

Note that the above description is not limited to a case in which one measurement region is set in one bronchial region, and two measurement regions inscribed in a luminal region and circumscribing the luminal region may be set.

To check whether a measurement region is set at a correct position, the display circuitry 19 preferably display a plurality of slice images in a plurality of phases while explicitly showing the measurement region. Upon determining that the position of the measurement region is not correct, the user adjusts the measurement region via the input circuitry 21. Upon determining that the position of the measurement region is correct, the user issues an instruction to execute the subsequent processing via the input circuitry 21. In response to the input of the instruction via the input circuitry 21, the image processing circuitry 17 perform the subsequent processing. Note that when the image processing circuitry 17 set a measurement region, the subsequent processing may be automatically performed.

When a measurement region is set over a plurality of phases, the image processing circuitry 17 measure the morphological index value of the bronchial region in each phase. Morphological index values of a bronchial region include, for example, a luminal area, the length of an inner circumference, the length of an outer circumference, the length of a long axis, the length of a short axis, the ratio between the length of the long axis and the length of the short axis, and the degree of circularity. More specifically, a luminal area is measured as the area of the measurement region, the length of an inner circumference is measured as the length of the measurement region inscribed in the luminal region in the bronchial region, the length of an outer circumference is measured as the length of the measurement region circumscribing the luminal region of the bronchial region, the length of a long axis is measured as the length of the long axis of the measurement region, the length of a short axis is measured as the length of the short axis of the measurement region, and the degree of circularity is measured as the degree of circularity of the measurement region.

Upon execution of step SA2, the control circuitry 11 cause the image processing circuitry 17 to execute the infiltration determination function 175 (step SA3). In step SA3, the image processing circuitry 17 determine whether esophagus cancer has infiltrated the bronchi, based on a change in morphological index value over a plurality of phases.

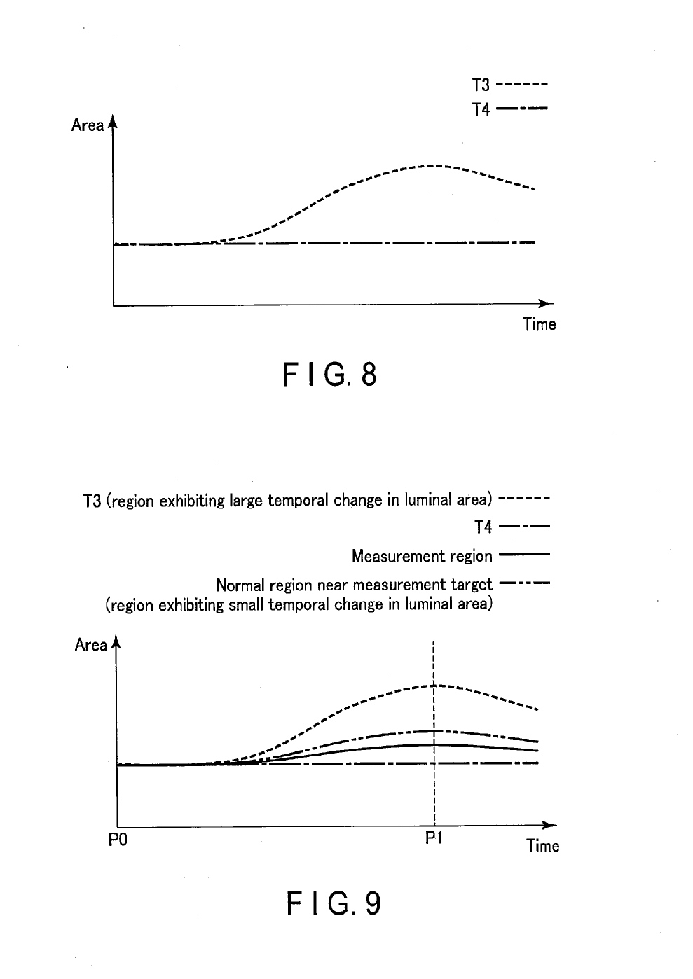

The relationship between a change in morphological index value and the infiltration of esophagus cancer into the bronchi will be described with reference to FIGS. 7 and 8. FIG. 7 schematically shows a temporal change in the form of the bronchial region RA in a measurement target slice at each of stages T3 and T4. FIG. 8 is a graph showing a temporal change in luminal area as one of the morphological index values at each of stages T3 and T4. The ordinate of FIG. 8 defines the luminal area, and the abscissa defines the time. At stage T3, since esophagus cancer has not infiltrated the surrounding bronchi, the bronchi expands and contracts in accordance with the respiration of the object. For this reason, at stage T3, as shown in FIGS. 7 and 8, the form of the bronchial region RA in the measurement target slice changes with the lapse of time. In contrast to this, at stage T4, since esophagus cancer has infiltrated the bronchi, the infiltrated bronchi become hardened. For this reason, the bronchi around esophagus cancer do not expand or contract regardless of the respiration of the object. That is, at stage T4, as shown in FIGS. 7 and 8, the form of the bronchial region RA in the measurement target slice does not change with the lapse of time.

The image processing circuitry 17 determine whether esophagus cancer has infiltrated the bronchial tissue, by using the difference between the form of a change in morphological index value at T3 and the form of a change in morphological index value at T4. For example, the image processing circuitry 17 determine whether esophagus cancer has infiltrated the bronchial tissue, based on the difference between a morphological index value in a reference phase P0 and a morphological index value in a comparative phase P1 which are measured in step SA2. The reference phase P0 and the comparative phase P1 are preferably set to phases in which the degree of expansion of the bronchi and the degree of contraction of the bronchi are maximum. For example, the reference phase P0 is preferably set to a phase corresponding to an expiration, and the comparative phase P1 is preferably set to a phase corresponding to an inspiration. The difference between a morphological index value in the reference phase P0 and a morphological index value in the comparative phase P1 concerning T4 and T3 exhibits a significant difference as compared with the difference between a morphological index value in the reference phase P0 and a morphological index value in the comparative phase P1 concerning T4.

The image processing circuitry 17 execute the infiltration determination function 175 to calculate the difference between a morphological index value in the reference phase P0 and a morphological index value in the comparative phase P1 and compare the calculated difference with a preset threshold. The threshold is preferably set to a value smaller than a standard difference at T3 and larger than a standard difference at T4. If the difference is smaller than the threshold, the image processing circuitry 17 determine that the esophagus cancer has not infiltrated the bronchi. If the difference is larger than the threshold, the image processing circuitry 17 determine that the esophagus cancer has infiltrated the bronchi.

Note that the degree of expansion/contraction of the bronchi sometimes differs depending on the anatomical position of a bronchial portion as a measurement target as well as the degree of esophagus cancer infiltration. For example, a bronchial portion distant from the heart is less susceptible to pulsation than a bronchial portion near to the heart. For this reason, a change in morphological index value with the lapse of time is sometimes relatively small even at T3. It is also possible to perform determination processing in consideration of the tendency of a change in morphological index value in accordance with such an anatomical position.

For example, the image processing circuitry 17 may determine whether esophagus cancer has infiltrated the bronchi, based on the comparison between a change in morphological index value of a measurement region and a change in morphological index value of a normal region. FIG. 9 is a graph showing a temporal change in luminal area as one of the morphological index values of a measurement region and a normal region at T4. FIG. 9 also shows, as a comparative example, a temporal change in T3 concerning a region exhibiting an anatomically relatively large temporal change in luminal area. It is preferable to set a normal region in a tubular tissue region portion corresponding to a region, of anatomically the same tubular tissue as that in which a measurement region is set, which esophagus cancer has not infiltrated with high likelihood. For example, when a measurement region is set in the bronchial region, a normal region is preferably set in a bronchi region portion corresponding to a bronchial portion which esophagus cancer has not clearly infiltrated. In addition, to reduce the difference between changes in morphological index value of a measurement region and a normal region due to the difference in anatomical position, the normal region is preferably set in a region as near as possible to a region in which the measurement region is set. The image processing circuitry 17 execute the measurement function 173 to set a normal region together with a measurement region. In this case, the image processing circuitry 17 may set a normal region in accordance with an input from the user via the input circuitry 21 or may be automatically set by image processing. When setting a normal region by image processing, the image processing circuitry 17 set, for example, a plurality of orthogonal slices along the bronchial region, measures the shape of the bronchial region concerning each orthogonal slice, and sets a bronchial region having an almost elliptic shape as the normal region.

In this case, the image processing circuitry 17 execute the infiltration determination function 175 to calculate the difference between a morphological index value in the reference phase P0 and a morphological index value in the comparative phase P1 concerning the normal region, calculates the difference between a morphological index value in the reference phase P0 and a morphological index value in the comparative phase P1 concerning the measurement region, and compares the difference associated with the normal region with the difference associated with the measurement region. Upon determining that the difference associated with the measurement region is a significant difference with respect to the difference associated with the normal region, the image processing circuitry 17 determine that esophagus cancer has infiltrated the bronchi. Upon determining that the difference associated with the measurement region is not a significant difference with respect to the difference associated with the normal region, the image processing circuitry 17 determine that esophagus cancer has not infiltrated the bronchi.

Upon execution of step SA3, the control circuitry 11 cause the display circuitry 19 to perform display processing (step SA4). In step SA4, the display circuitry 19 display the determination result obtained in step SA3.

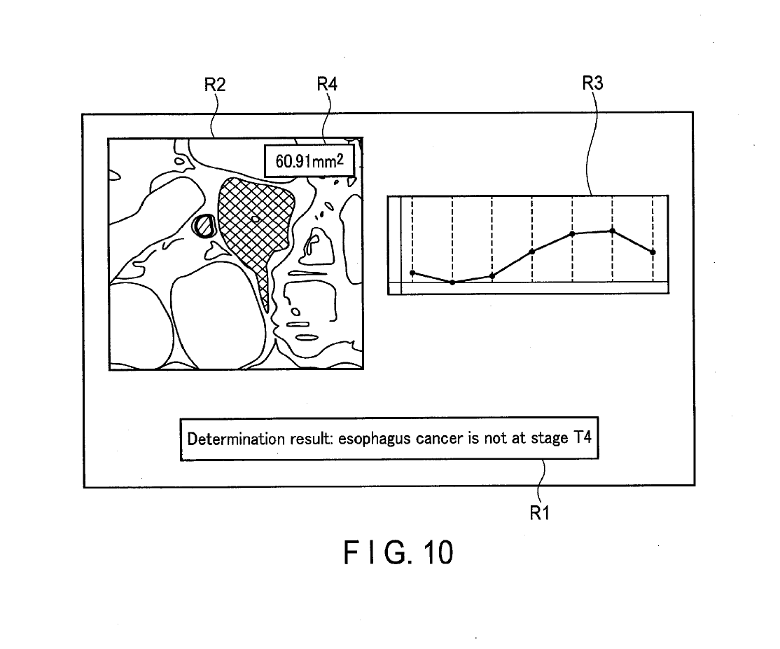

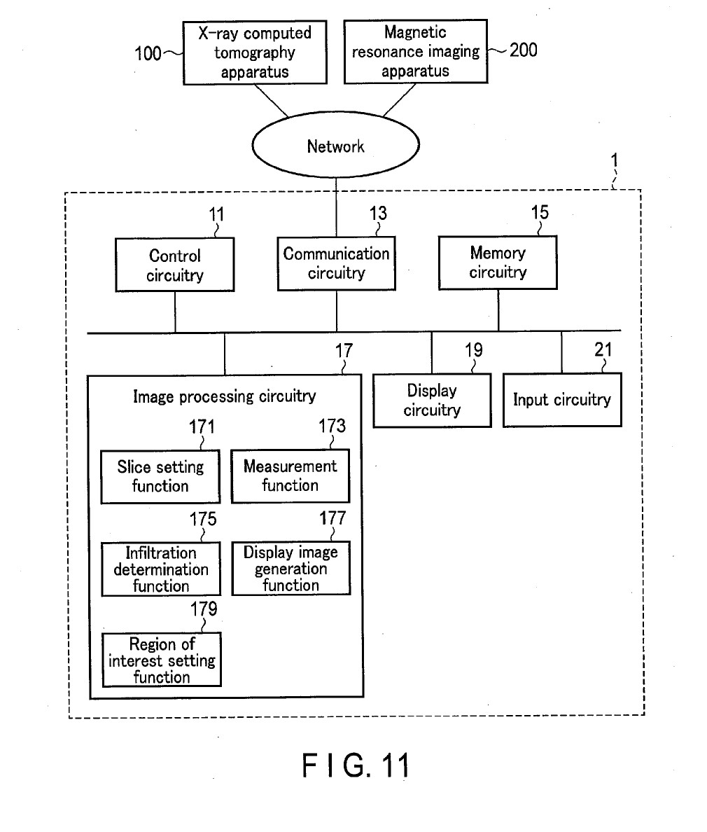

FIG. 10 is a view showing a display example of the determination result displayed by the display circuitry 19. As shown in FIG. 10, the display screen includes a determination result display area R1, an image display area R2, a graph display area R3, and a morphological measurement value display area R4. The determination result obtained in step S3 is displayed in the determination result display area R1. For example, if the infiltration determination function 175 determines that esophagus cancer has not infiltrated the bronchi, the display circuitry 19 display a corresponding message, for example, “esophagus cancer is not at stage T4”, as shown in FIG. 10. If the infiltration determination function 175 determines that esophagus cancer has infiltrated the bronchi, the display circuitry 19 display a corresponding message, for example, “esophagus cancer is at stage T4”. This allows the user to check whether the esophagus cancer is at stage T4, in other words, whether the esophagus cancer has infiltrated the bronchi. A slice image of a measurement target slice is displayed in the image display area R2. For example, the display circuitry 19 dynamically display a slice image over a plurality of slices. Note that an image to be displayed in the image display area R2 is not limited to a slice image of a measurement target slice, and may be any type of image based on a plurality of three-dimensional images in a plurality of phases. The morphological measurement value measured in step SA2 is displayed in the morphological measurement value display area R4. The display circuitry 19 preferably display a morphological measurement value together with a slice image of a measurement target slice. For example, it is preferable that the display circuitry 19 dynamically superimpose and displays a morphological measurement value and a slice image of a measurement target slice upon matching the phases. This allows the user to grasp the relationship between the form of a bronchial region and a morphological measurement value. Note that a morphological measurement value and a slice image may be displayed side by side. A graph indicating a change in the morphological measurement value of a measurement region measured in step SA2 is displayed in the graph display area R3. Displaying a slice image of a measurement target slice and a graph indicating a change in morphological measurement value as well as a determination result in this manner allows the user to determine the reliability of the determination result.

Upon execution of step SA4, the infiltration determination processing performed under the control of the control circuitry 11 are terminated.

Note that in this embodiment, a measurement region is set in a bronchial region. However, the embodiment is not limited to this. For example, a measurement region may be set in a blood vessel with likelihood of esophagus cancer infiltration, for example, an image region of a large vessel (to be referred to as a large vessel region hereinafter).

In addition, in this embodiment, a measurement region is set at one position in one tubular tissue region. However, the embodiment is not limited to this. Measurement regions may be set at a plurality of positions in one tubular tissue region. In this case, the image processing circuitry 17 execute the measurement function 173 to measure a morphological measurement value of each of a plurality of measurement regions over a plurality of phases. The image processing circuitry 17 individually determine, based on a change in morphological measurement value, whether esophagus cancer has infiltrated the tubular tissue in each of the plurality of measurement regions. Upon determining that there is at least one measurement region which esophagus cancer has infiltrated, the image processing circuitry 17 determine that the esophagus cancer has infiltrated the tubular tissue. In contrast to this, upon determining that there is no measurement region which the esophagus cancer has infiltrated, the image processing circuitry 17 determine that the esophagus cancer has not infiltrated the tubular tissue. Note that upon determining that there is at least one measurement region which the esophagus cancer has infiltrated, the image processing circuitry 17 may determine that the esophagus cancer has infiltrated the tubular tissue, whereas upon determining that there is no measurement region which the esophagus cancer has infiltrated, the image processing circuitry 17 may determine that the esophagus cancer has not infiltrated the tubular tissue. In this manner, setting a plurality of measurement regions in one tubular tissue region makes it possible to more accurately determine whether the esophagus cancer has infiltrated the tubular tissue.

In addition, in this embodiment, a measurement region is set in one tubular tissue. However, the embodiment is not limited to this. Measurement regions may be set in a plurality of tubular tissues. For example, the image processing circuitry 17 may execute the measurement function 173 to individually set measurement regions in a bronchial region and a large blood vessel region. In this case, the image processing circuitry 17 measure morphological measurement values of the bronchial region and the large blood vessel region over a plurality of phases. The image processing circuitry 17 execute the infiltration determination function 175 to determine, based on a change in the morphological measurement value of the bronchial region, whether esophagus cancer has infiltrated the bronchi, and separately determines, based on a change in the morphological measurement value of the large vessel region, whether the esophagus cancer has infiltrated the large vessel. Upon determining that the esophagus cancer has infiltrated at least one of the bronchi and the large blood vessel, the image processing circuitry 17 determine that the esophagus cancer is at stage T4. Upon determining that the esophagus cancer has infiltrated neither of the bronchi nor the large blood vessel, the image processing circuitry 17 determine that the esophagus cancer is not at stage T4. Setting a plurality of measurement regions in a plurality of tubular tissue regions in this manner makes it possible to more accurately determine whether esophagus cancer has infiltrated a surrounding tubular tissue.

As described above, the medical image processing apparatus 1 according to the first embodiment provides a new determination method concerning the presence/absence of infiltration of esophagus cancer into a tubular tissue based on a characteristic that the form of a tubular tissue changes with time differently depending on whether esophagus cancer has infiltrated a surrounding tubular tissue.

For this purpose, the medical image processing apparatus 1 according to the first embodiment includes the image processing circuitry 17. The image processing circuitry 17 include the slice setting function 171, the measurement function 173, and the infiltration determination function 175. The image processing circuitry 17 execute the slice setting function 171 to set a measurement target slice intersecting with a tubular tissue region of a tubular tissue with likelihood of tumor infiltration with respect to a plurality of three-dimensional images in a plurality of phases. The image processing circuitry 17 execute the measurement function 173 to measure a morphological index value on a measurement target slice of a tubular tissue region over a plurality of phases. The image processing circuitry 17 execute the infiltration determination function 175 to determine, based on a change in morphological index value over a plurality of phases, whether a tumor has infiltrated the tubular tissue.

With the above arrangement, this method can more accurately determine whether a tumor has infiltrated a tubular tissue than the conventional determination method. A user such as a doctor can therefore more accurately determine, by giving consideration to this determination result, whether it is possible to surgically remove esophagus cancer. More specifically, even if the conventional determination method erroneously diagnoses a given case as T4 and excludes it from surgical treatment, the method according to the first embodiment can correctly diagnose the case as T3.

That is, according to the first embodiment, it is possible to more accurately determine the infiltration of a tumor into a tubular tissue.

### Second Embodiment

A medical image processing apparatus according to the second embodiment will be described next. Note that the same reference numerals in the following description denote constituent elements having almost the same functions and arrangements as those of the first embodiment, and a repetitive description will be made only when required.

FIG. 11 is a block diagram showing the arrangement of a medical image processing apparatus 1 according to the second embodiment. As shown in FIG. 11, image processing circuitry 17 according to the second embodiment implement a slice setting function 171, a measurement function 173, a infiltration determination function 175, a display image generation function 177, and a region-of-interest setting function 179 by executing image processing programs for infiltration determination according to the second embodiment.

The image processing circuitry 17 execute the slice setting function 171 to set a first ROI (Region Of Interest) and a second ROI with respect to a plurality of three-dimensional images in a plurality of phases. The first ROI is set in a pixel region (to be referred to as the first peripheral portion region hereinafter) of the first peripheral portion with likelihood of tumor infiltration. When the tumor is esophagus cancer, the first peripheral portion with likelihood of tumor infiltration includes the bronchi, aorta, and lymph node located near the tumor. The second ROI is set in an image region (to be referred to as the second peripheral portion region hereinafter) of the second peripheral portion different from the first peripheral portion. The second peripheral portion region is preferably, for example, an anatomical region adjacent to the bronchial region in which the first ROI is set. Such an anatomical region is preferably, for example, an aorta region or lymph node region adjacent to bronchial region when the first ROI is set in the bronchial region. Note that the second ROI may be or may not be in contact with the first ROI as long as the second ROI does not overlap the first ROI.

The image processing circuitry 17 execute the measurement function 173 to measure index values indicating the position variations of the first ROI and the second ROI. The index values will be referred to as position variation amounts hereinafter. More specifically, the image processing circuitry 17 measure the position variation amount of the first ROI and the position variation amount of the second ROI, and calculates the degree of similarity in position variation amount between the first ROI and the second ROI.

The image processing circuitry 17 execute the infiltration determination function 175 to determine whether the tumor has infiltrated the first peripheral portion and the second peripheral portion, based on the position variations of the first ROI and the second ROI over at least two phases of a plurality of phases. Display circuitry 19 display the determination result.

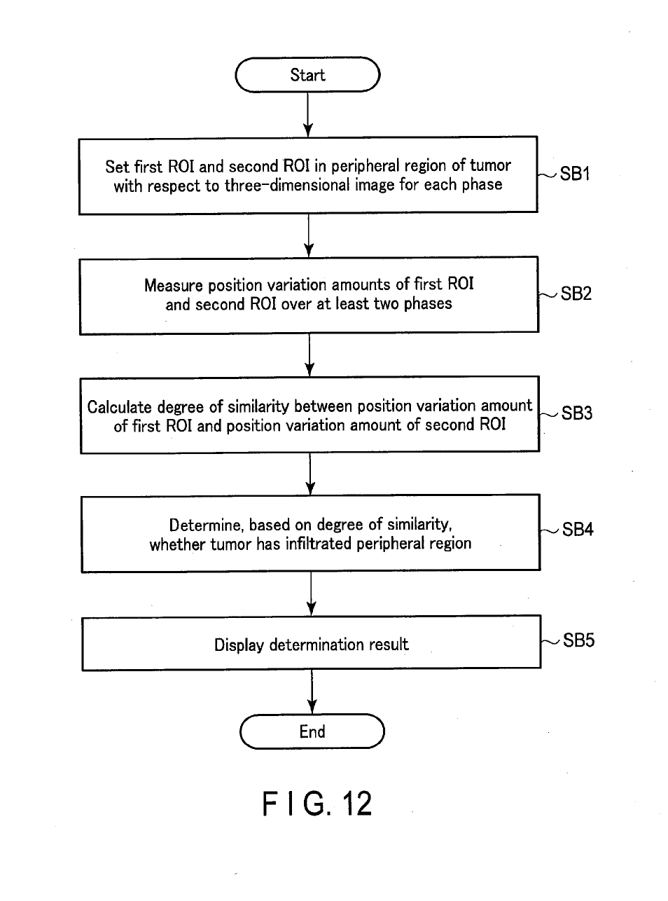

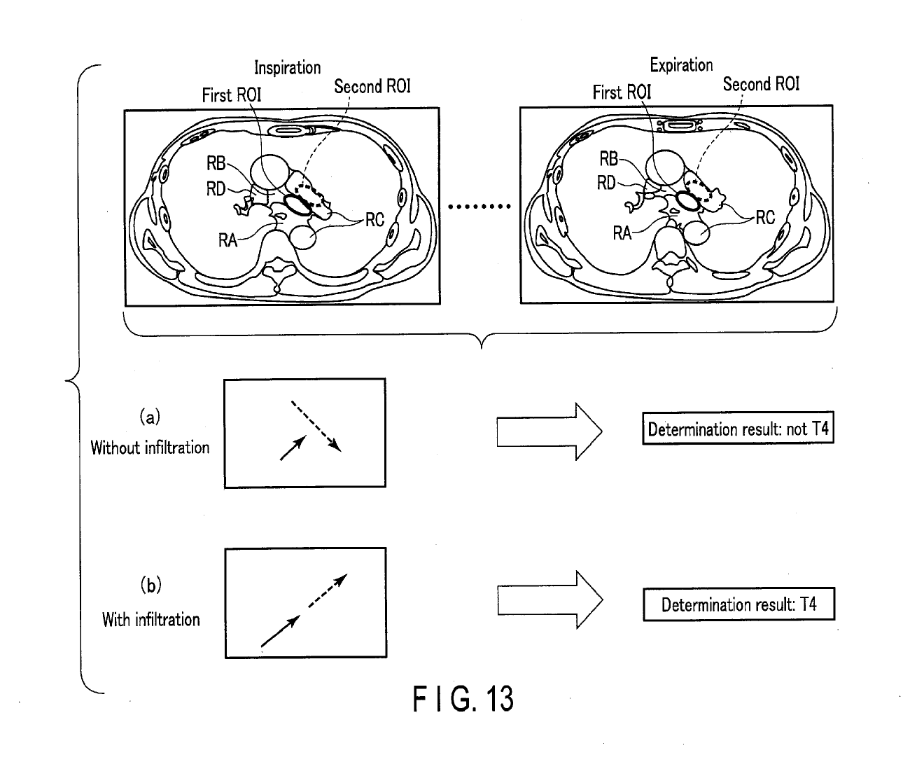

FIG. 12 is a flowchart showing a procedure for infiltration determination processing to be performed under the control of control circuitry 11 according to the second embodiment. FIG. 13 is a view schematically showing a procedure for infiltration determination processing according to the second embodiment. As shown in FIG. 12, first of all, the control circuitry 11 cause the image processing circuitry 17 to execute the region-of-interest setting function 179 (step SB1). In step SB1, the image processing circuitry 17 set the first ROI and the second ROI in a peripheral portion of a tumor with respect to a three-dimensional image for each phase.

For example, as shown in FIG. 13, the first ROI is set in a tubular tissue region RB of a tubular tissue with likelihood of tumor infiltration, and the second ROI is set in an image region (to be referred to as an aorta region hereinafter) RC of the aorta adjacent to the tubular tissue. Note that the tubular tissue region RB exists around an image region (tumor region) RA of the tumor. In addition to the tubular tissue region RB, the aorta region RC and an image region (lymph node region) RD of the lymph node exist around the tumor region RA. The first ROI may be set in any place as long as it is an anatomical region of an anatomical tissue with likelihood of tumor infiltration. More specifically, the first ROI may be set in any of the following: the tubular tissue region RB, the aorta region RC, the lymph node region RD, and the like. The second ROI is preferably set in an anatomical region adjacent to the anatomical region in which the first ROI is set. As shown in FIG. 13, when the first ROI is set in the tubular tissue region RB, the second ROI can be set in the aorta region RC or the lymph node region RD adjacent to the tubular tissue region RB.

Note that the first ROI and the second ROI may be set with respect to all phases or may be set with respect to limited phases in which position variation amounts should be measured. The user can arbitrarily set, via input circuitry 21, a phase as an ROI setting target.

Note that the image processing circuitry 17 may set the first ROI and the second ROI as three-dimensional regions or two-dimensional regions. For example, when the positions of the first ROI and the second ROI are manually designated on a measurement slice of a three-dimensional image in each phase via the input circuitry 21 or the like, the first ROI and the second ROI are preferably set in two-dimensional regions including the designated positions. In this case, the position variations of the first ROI and the second ROI on the measurement slice are measured. In the following description, however, for simpler and more accurate infiltration determination, assume that the first ROI and the second ROI have three-dimensional regions. When, for example, the positions of the first ROI and the second ROI are manually designated on a measurement slice of a three-dimensional image in each phase via the input circuitry 21 or the like, the first ROI and the second ROI are set in three-dimensional regions including the designated positions. Alternatively, three-dimensional anatomical regions corresponding to the designated positions may be segmented from a three-dimensional image by image processing, and the first ROI and the second ROI may be set in the anatomical regions.

Upon execution of step SB1, the control circuitry 11 cause the image processing circuitry 17 to execute the measurement function 173 (step SB2). In step SB2, the image processing circuitry 17 measure the position variation amounts of the first ROI and the second ROI over at least two phases. A position variation amount is, for example, the movement amount of the first ROI or second ROI between the first phase and the second phase. Note that a movement amount according to the second embodiment may be, for example, a movement distance or moving direction or a movement vector as a combination of a movement distance and a moving direction. Assume that in the following description, a position variation amount is a movement vector.

For example, the image processing circuitry 17 specify the positions of the first ROI and the second ROI by tracking the first ROI and the second ROI over a plurality of phases by image processing. The image processing circuitry 17 then preferably measure the movement vector of each of the first ROI and the second ROI over two arbitrary phases of the plurality of phases. Two phases over which each movement vector should be measured are preferably set to two phases over which the position varies relatively noticeably. As shown in FIG. 13, such phases include an inspiration phase and an expiration phase. The image processing circuitry 17 measure the movement vector of the first ROI over an inspiration phase and an expiration phase. More specifically, the image processing circuitry 17 measure the movement vector of each pixel of the first ROI, that is, the movement distance and the moving direction of each pixel, as a movement vector. The image processing circuitry 17 then calculate the statistical value of the movement vectors of the respective pixels and sets the statistic value as the movement vector of the first ROI. A statistic value is, for example, the average value, mode value, median value, maximum value, or minimum value of the movement vectors of the respective pixels. The image processing circuitry 17 measure the movement vector of the second ROI in the same manner as described above.

Note that the method of measuring the movement vectors of the first ROI and the second ROI is not limited to the above method. For example, the image processing circuitry 17 may measure the movement vector of a reference pixel of each ROI as the movement vector of each ROI. It is possible to set, as a reference pixel, a pixel, of the pixels constituting each ROI, which corresponds to the center, the barycenter, or an end point, or an arbitrary pixel designated by the user.

Upon execution of step SB2, the image processing circuitry 17 calculate the degree of similarity in position variation amount between the first ROI and the second ROI (step SB3). As the degree of similarity, an arbitrary index value indicating the difference between the movement vector of the first ROI and the movement vector of the second ROI is used. For example, the image processing circuitry 17 calculate, as the degree of similarity, the difference between the movement vector of the first ROI and the movement vector of the second ROI. A smaller difference indicates a larger degree of similarity, and vice versa. Note that a movement vector has a movement distance and a moving direction as components. For this reason, the image processing circuitry 17 calculate the difference between the movement distance of the first ROI and the movement amount of the second ROI and the difference between the moving direction of the first ROI and the moving direction of the second ROI.

Upon execution of step SB3, the control circuitry 11 execute the infiltration determination function 175 (step SB4). In step SB4, the image processing circuitry 17 determine, based on the degree of similarity, whether the tumor has infiltrated the peripheral portion.

Determination processing will be described in detail below with reference to FIG. 13. Assume that the first ROI is set in the bronchial region, and the second ROI is set in the aorta region adjacent to the bronchial region. As described above, the degree of similarity indicates the difference between the movement vector of the first ROI and the movement vector of the second ROI, and is, for example, the difference between the movement vector of the first ROI and the movement vector of the second ROI. In other words, the degree of similarity indicates the evaluation of the similarity between the movement of a tissue corresponding to the first ROI and the movement of a tissue corresponding to the second ROI accompanying respiratory movement.

As indicated by (a) in FIG. 13, when esophagus cancer has not infiltrated the bronchi and the aorta, that is, is not at stage T4, the bronchi and the aorta independently move. Therefore, the degree of similarity in movement vector between the first ROI and the second ROI is relatively low. In contrast to this, as indicated by (b) in FIG. 13, when the esophagus cancer has infiltrated the bronchi and the aorta, that is, is at stage T4, the independence of the bronchi and the aorta is lost, and hence the bronchi and the aorta move dependently of each other. Therefore, the degree of similarity in movement vector between the first ROI and the second ROI is relatively high.

By using the difference between the movements of such tissues around a tumor, the image processing circuitry 17 determine whether esophagus cancer has infiltrated the peripheral tissues. More specifically, the image processing circuitry 17 compare the degree of similarity with a preset threshold. The threshold is preferably set to a value that makes it possible to discriminate a value that the degree of similarity can take when esophagus cancer has infiltrated the first and second peripheral portions from a value that the degree of similarity can take when the esophagus cancer has not infiltrated the first and second peripheral portions. The threshold can be set to an arbitrary value by the user or the like via the input circuitry 21 or the like. Alternatively, the threshold may be set to a value that differs depending on the type and location of an anatomical region in which each ROI is set.

If the degree of similarity is lower than the threshold, the image processing circuitry 17 determine that the esophagus cancer has not infiltrated the first and second peripheral portions, that is, is not at stage T4. If the degree of similarity is higher than the threshold, the image processing circuitry 17 determine that the esophagus cancer has infiltrated the first and second peripheral portions, that is, is at stage T4.

Upon execution of step SB4, the control circuitry 11 cause the display circuitry 19 to perform display processing (step SB5). In step SB5, the display circuitry 19 display the determination result obtained in step SB4. For example, as shown in FIG. 13, if the control circuitry 11 determine that the esophagus cancer has not infiltrated the bronchi and the aorta, that is, is not at stage T4, the display circuitry 19 display a corresponding message, e.g., “esophagus cancer is not at stage T4”. If the control circuitry 11 determine that the esophagus cancer has infiltrated the bronchi and the aorta, that is, is at stage T4, the display circuitry 19 display a corresponding message, e.g., “esophagus cancer is at stage T4”. This allows the user to determine whether the esophagus cancer is at stage T4, in other words, the esophagus cancer has infiltrated the bronchi and the aorta.

In this case, the display circuitry 19 may display the degree of similarity and a slice image of a measurement target slice passing through the first ROI and the second ROI, in addition to the determination result. This slice image may be generated by the image processing circuitry 17 based on a three-dimensional image. Displaying a determination result together with a degree of similarity and a slice image allows the user to determine the reliability of the determination result.

Upon execution of step SB5, the infiltration determination processing performed under the control of the control circuitry 11 is terminated.

As described above, the medical image processing apparatus 1 according to the second embodiment includes the image processing circuitry 17 which processes a plurality of three-dimensional images in a plurality of phases. The image processing circuitry 17 include the region-of-interest setting function 179 and the infiltration determination function 175. The image processing circuitry 17 execute the region-of-interest setting function 179 to set the first ROI including the first peripheral portion region of the first peripheral portion with likelihood of tumor infiltration with respect to a plurality of three-dimensional images in a plurality of phases and the second ROI including the second peripheral portion region of the second peripheral portion different from the first peripheral portion. The image processing circuitry 17 execute the infiltration determination function 175 to determine whether a tumor has infiltrated the first peripheral portion and the second peripheral portion, based on the position variations of the first ROI and the second ROI over at least two phases of the plurality of phases.

With the above arrangement, the medical image processing apparatus 1 according to the second embodiment can determine, by using the difference between the movements of peripheral tissues of a tumor, whether esophagus cancer has infiltrated the peripheral tissues. With this operation, even if the form of a peripheral tissue of a tumor does not change with the lapse of time, it is possible to determine whether the tumor has infiltrated the peripheral tissue and the other peripheral tissue.

As described above, according to the second embodiment, it is possible to more accurately determine whether a tumor has infiltrated a tubular tissue.

### Third Embodiment

A medical image processing apparatus according to the third embodiment will be described next. The second embodiment described above is configured to determine whether a tumor has infiltrated a peripheral tissue, based on the degree of similarity between the position variation amounts of the movement vectors of the first ROI and the second ROI. Assume, however, that the body motion of an overall portion in which the first ROI and the second ROI are set is dominant. In this case, even if the position variation amount of the movement vector of the first ROI is similar to that of the second ROI, it is hard to say that the tumor has infiltrated the peripheral tissue. For this reason, the medical image processing apparatus according to the third embodiment is configured to set an ROI (big ROI) covering the first ROI and the second ROI and observe the dynamic state of the big ROI, thereby improving the reliability and accuracy of infiltration determination. Note that the same reference numerals in the following description denote constituent elements having almost the same functions and arrangements as those of the second embodiment, and a repetitive description will be made only when required.

Image processing circuitry 17 according to the third embodiment implement a slice setting function 171, a measurement function 173, an infiltration determination function 175, a display image generation function 177, and a region-of-interest setting function 179 by executing image processing programs for infiltration determination according to the third embodiment.

The image processing circuitry 17 execute the slice setting function 171 to set the first ROI and the second ROI in a plurality of three-dimensional images in a plurality of phases. The first ROI is set in a peripheral portion region of a peripheral portion with likelihood of tumor infiltration. The peripheral portion according to the third embodiment is the same as that according to the second embodiment, and hence a description of it will be omitted. The second ROI is set so as to cover the first ROI. That is, the second ROI has a larger volume than the first ROI. The first ROI and the second ROI will be respectively referred to as the small ROI and the big ROI hereafter. The number of small ROIs may be one or more. If a plurality of small ROIs are set, the big ROI is preferably set so as to cover all the small ROIs. The respective small ROIs are preferably set in different anatomical regions around a tumor, like the first ROI and the second ROI in the second embodiment.

The image processing circuitry 17 execute the measurement function 173 to measure position variation amounts representing the position variations of the small ROI and the big ROI. More specifically, the image processing circuitry 17 measure the position variation amount of the small ROI and the position variation amount of the big ROI, and calculate the degree of similarity in position variation amount between the small ROI and the big ROI.

The image processing circuitry 17 execute the infiltration determination function 175 to determine whether the tumor has infiltrated the peripheral portion in which the small ROI is set, based on the position variations of the small ROI and the big ROI over at least two phases of the plurality of phases. Display circuitry 19 display the determination result.

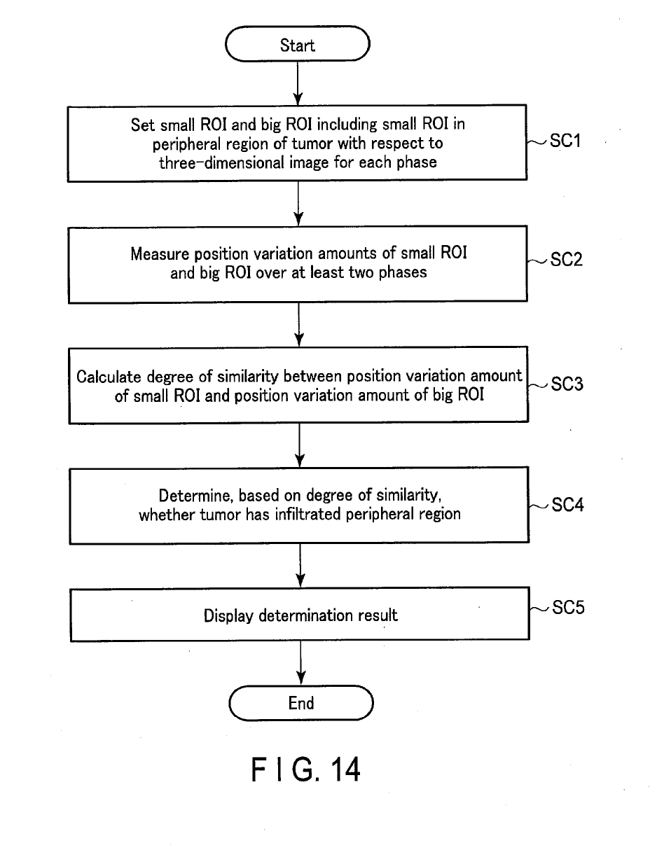

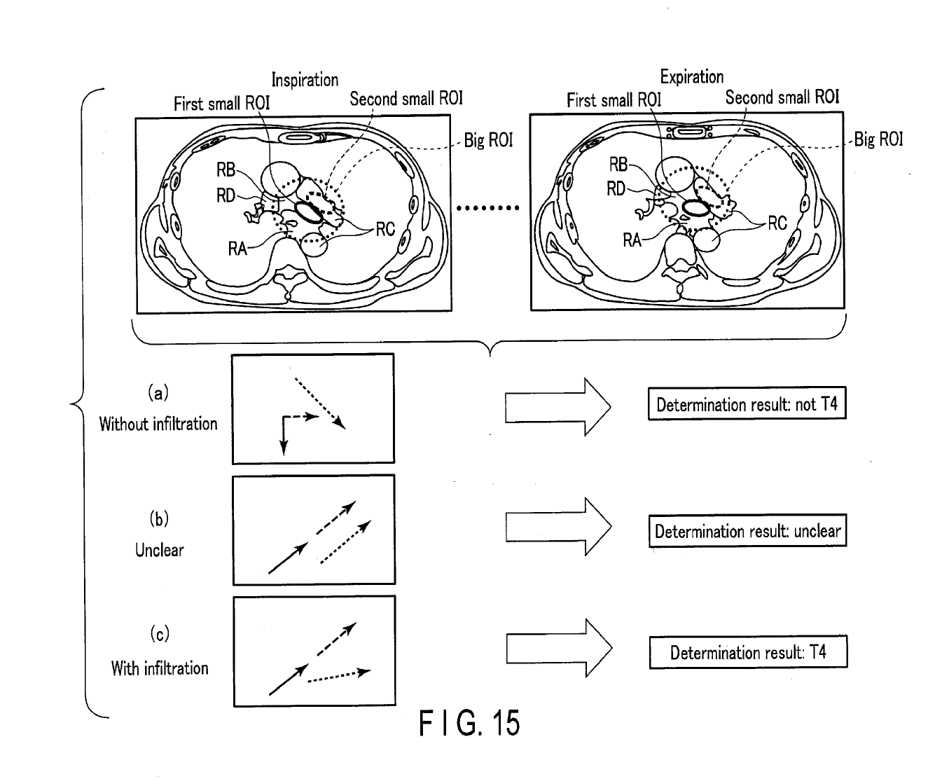

FIG. 14 is a flowchart showing a procedure for infiltration determination to be performed under the control of control circuitry 11 according to the third embodiment. FIG. 15 is a view schematically showing a procedure for infiltration determination processing according to the third embodiment. As shown in FIG. 14, first of all, the control circuitry 11 cause the image processing circuitry 17 to execute the region-of-interest setting function 179 (step SC1). In step SC1, the image processing circuitry 17 set a small ROI and a big ROI including the small ROI in peripheral portions of the tumor with respect to a three-dimensional image for each phase.

For example, as shown in FIG. 15, the small ROI is set in an anatomical region located at a periphery of the tumor and corresponding to an anatomical tissue with likelihood of tumor infiltration. The number of small ROIs may be one or more. Assume that in the following description, the number of small ROIs is two. The big ROI is set so as to cover the first small ROI and the second small ROI. The small ROI is set to check the local dynamic state of an anatomical region in which the small ROI is set. The big ROI is set to check the global dynamic state of a region including a plurality of anatomical regions in which a plurality of small ROIs are set. For example, the first small ROI is set in a tubular tissue region RB corresponding to a tubular tissue with likelihood of tumor infiltration. The second small ROI is set in an aorta region RC corresponding to the aorta adjacent to the tubular tissue. The big ROI is set in a chest region including the tubular tissue region RB and the aorta region RC. In this case, the first small ROI makes it possible to check the dynamic state of the tubular tissue, the second small ROI makes it possible to check the dynamic state of the aorta, and the big ROI makes it possible to check the dynamic state of the chest portion.

Small ROIs and big ROIs may be set with respect to all phases or may be set with respect to limited phases in which position variation amounts should be measured. The user can arbitrarily set, via the input circuitry 21, a phase as an ROI setting target. Alternatively, as in the second embodiment, the image processing circuitry 17 may set small ROIs and a big ROI as three-dimensional regions or two-dimensional regions.

Upon execution of step SC1, the control circuitry 11 cause the image processing circuitry 17 to execute the measurement function 173 (step SC2). In step SC2, the image processing circuitry 17 measure the position variation amounts of the small ROIs and the big ROI over at least two phases. Assume that position variation amounts are, for example, the movement vectors of the small ROIs and the big ROI between the first phase and the second phase, as in the second embodiment.

Upon execution of step SC2, the image processing circuitry 17 calculate the degree of similarity in position variation amount between each small ROI and the big ROI (step SC3). As a degree of similarity, an arbitrary index value indicating the difference between the movement vector of the small ROI and the movement vector of the big ROI is used, as in the second embodiment. For example, the image processing circuitry 17 calculate the difference between the movement vector of each small ROI and the movement vector of the big ROI as a degree of similarity. Note that the degree of similarity in movement vector among the first small ROI, the second small ROI, and the big ROI is preferably defined as the statistical value of at least two differences among the difference between the movement vector of the first small ROI and the movement vector of the big ROI, the difference between the movement vector of the second small ROI and the movement vector of the big ROI, and the difference between the movement vector of the first small ROI and the movement vector of the second small ROI. A statistic value is, for example, an average value, sum value, integral value, maximum value, minimum value, or the like.

Upon execution of step SC3, the control circuitry 11 execute the infiltration determination function 175 (step SC4). In step SC4, the image processing circuitry 17 determine, based on the degree of similarity, whether the tumor has infiltrated the peripheral portion.

Determination processing will be described in detail below with reference to FIG. 15. As indicated by (a) in FIG. 15, when the esophagus cancer has not infiltrated the bronchi and the aorta, that is, is not at stage T4, the bronchi and the aorta and the chest portion including the bronchi and the aorta independently move. Therefore, the degree of similarity in movement vector between the first small ROI, the second small ROI, and the big ROI is relatively low. In this case, it is possible to determine the presence/absence of infiltration based on the degree of similarity in movement vector between the first small ROI and the second small ROI without giving any consideration to the movement vector of the big ROI.

When the degree of similarity in movement vector among the first small ROI, the second small ROI, and the big ROI is high as indicated by (b) in FIG. 15, since the movement vector of the first small ROI is similar to that of the second small ROI, it may be thought that the esophagus cancer has infiltrated the bronchi and the aorta. However, since the movements of the bronchi and the aorta are similar to that of the chest portion including them, the movements of the bronchi and the aorta also seem to originate from body motion. When, therefore, the degree of similarity in movement vector among the first small ROI, the second small ROI, and the big ROI is high, it is difficult to determine the presence/absence of infiltration.

In contrast to this, as indicated by (c) in FIG. 15, when the movement vector of the first small ROI is similar to that of the second small ROI and the movement vectors of the first small ROI and the second small ROI are not similar to the movement vector of the big ROI, it is thought that the esophagus cancer has infiltrated the bronchi and the aorta.

Based on such movements of peripheral tissues of a tumor and such movement of a global portion including the peripheral tissues, the image processing circuitry 17 determine whether the esophagus cancer has infiltrated the peripheral tissues. More specifically, the image processing circuitry 17 compare the degree of similarity with a preset threshold. For example, the image processing circuitry 17 compare the degree of similarity in movement vector among the first ROI, the second ROI, and the big ROI with the first threshold. For example, the first threshold is preferably set to a value that makes it possible to discriminate a value that the degree of similarity can take when esophagus cancer has not infiltrated the first and second peripheral portions from a value that the degree of similarity can take otherwise. The first threshold can be set to an arbitrary value by the user or the like via the input circuitry 21 or the like. Alternatively, the first threshold may be set to a value that differs depending on the type and location of an anatomical region in which each ROI is set.

If the degree of similarity in movement vector among the first ROI, the second ROI, and the big ROI is lower than the first threshold, the image processing circuitry 17 determine that the esophagus cancer has not infiltrated the first and second peripheral portions, that is, is not at stage T4. If the degree of similarity is higher than the first threshold, the image processing circuitry 17 determine that the esophagus cancer has not infiltrated the first and second peripheral portions, that is, is not at stage T4.

If the degree of similarity in movement vector among the first ROI, the second ROI, and the big ROI is higher than the first threshold, the image processing circuitry 17 compare the degree of similarity in movement vector among the first ROI, the second ROI, and the big ROI with the second threshold. For example, the second threshold is preferably set to a value that makes it possible to discriminate a value that the degree of similarity can take when the body motion is large from a value that the degree of similarity can take otherwise. The second threshold can be set to an arbitrary value by the user or the like via the input circuitry 21 or the like. Alternatively, the second threshold may be set to a value that differs depending on the type and location of an anatomical region in which each ROI is set.

If the degree of similarity in movement vector between the first ROI or second ROI and the big ROI is lower than the second threshold, the image processing circuitry 17 determine that the esophagus cancer has infiltrated the first and second peripheral portions, that is, is at stage T4. In contrast to this, if the degree of similarity in movement vector between the first ROI or second ROI and the big ROI is higher than the second threshold, the image processing circuitry 17 determine that the presence/absence of infiltration is unclear.

Upon execution of step SC4, the control circuitry 11 cause the display circuitry 19 to perform display processing (step SC5). In step SC5, the display circuitry 19 display the determination result obtained in step SC4. For example, as shown in FIG. 15, if the control circuitry 11 determine that the esophagus cancer has not infiltrated the bronchi and the aorta, that is, is not at stage T4, the display circuitry 19 display a corresponding message, e.g., “esophagus cancer is not at stage T4”. If the control circuitry 11 determine that the esophagus cancer has infiltrated the bronchi and the aorta, that is, is at stage T4, the display circuitry 19 display a corresponding message, e.g., “esophagus cancer is at stage T4”. If the control circuitry 11 determine that the presence/absence of infiltration is unclear, that is, whether the esophagus cancer is T4 is unclear, the display circuitry 19 display a corresponding message, e.g., “unclear”. This allows the display circuitry 19 to provide a determination result with higher credibility.

In this case, the display circuitry 19 may display the degree of similarity and a slice image of a measurement target slice passing through the small ROIs and the big ROI together with the determination result. This slice image may be generated by the image processing circuitry 17 based on a three-dimensional image. Displaying the determination result together with the degree of similarity and the slice image allows the user to determine the reliability of the determination result.

Upon execution of step SC5, the infiltration determination processing performed under the control of the control circuitry 11 is terminated.

Note that in the above embodiment, the image processing circuitry 17 set a plurality of small ROIs and a single big ROI. However, this embodiment is not limited to this. For example, the image processing circuitry 17 may set a single small ROI and a single big ROI. In this case, as in the second embodiment, the image processing circuitry 17 can determine, based on the degree of similarity in movement vector between the small ROI and the big ROI, whether a tumor has infiltrated a peripheral portion in which the small ROI is set.

In addition, in the above embodiment, the image processing circuitry 17 set a plurality of small ROIs and a big ROI at once. However, this embodiment is not limited to this. That is, first of all, upon determining, by executing infiltration determination processing according to the second embodiment, that the degree of similarity in movement vector between the first small ROI and the second small ROI is higher than a threshold, the image processing circuitry 17 may execute infiltration determination processing according to the third embodiment. Performing infiltration determination processing in this order allows the image processing circuitry 17 to determine that a tumor has not infiltrated a peripheral portion without setting any big ROI, when the tumor has not infiltrated the peripheral portion.

As described above, the medical image processing apparatus 1 according to the third embodiment includes the image processing circuitry 17 which processes a plurality of three-dimensional images in a plurality of phases. The image processing circuitry 17 include the region-of-interest setting function 179 and the infiltration determination function 175. The image processing circuitry 17 execute the region-of-interest setting function 179 to set a small ROI including the first peripheral portion region of a peripheral portion with likelihood of tumor infiltration and a big ROI including the small ROI. The image processing circuitry 17 execute the infiltration determination function 175 to determine whether the tumor has infiltrated the peripheral portion, based on the position variations of the small ROI and the big ROI over at least two phases of a plurality of phases.

With the above arrangement, the medical image processing apparatus 1 according to the third embodiment determines, by using the difference in movement between a small ROI locally set in a peripheral portion of a tumor and a big ROI globally set in the peripheral portion, whether the esophagus cancer has infiltrated the peripheral tissue. With this processing, even when the form of a peripheral tissue of a tumor does not change with the lapse of time, it is possible to determine whether the tumor has infiltrated the peripheral tissue and other peripheral tissues.

As has been described above, according to the third embodiment, it is possible to more accurately determine the infiltration of a tumor into a tubular tissue.

As described above, in the first, the second and the third embodiments, the tumor that is determination target of infiltration into the tubular tissue or the peripheral tissue is the esophagus cancer. However, this embodiment is not limited to this. The medical image processing apparatus 1 according to those embodiments may determines, same as the esophagus cancer, whether a lung cancer or cholangiocarcinoma (bile duct cancer) has infiltrated a peripheral tubular tissue and other peripheral tissues.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A medical image processing apparatus comprising image processing circuitry configured to process a plurality of three-dimensional images in a plurality of phases,
wherein the image processing circuitry set a slice intersecting with a tubular tissue region of a tubular tissue with likelihood of tumor infiltration with respect to the plurality of three-dimensional images in the plurality of phases,
measure a morphological index value on the slice of the tubular tissue region over the plurality of phases, and
determine, based on a change in the morphological index value over the plurality of phases, whether the tumor has infiltrated the tubular tissue.

2. The apparatus of claim 1, further comprising display circuitry configured to display a determination result indicating whether the tumor has infiltrated.

3. The apparatus of claim 1, further comprising display circuitry configured to display the morphological index value together with a display image based on the three-dimensional image.

4. The apparatus of claim 1, wherein the image processing circuitry determine, based on a comparison between a morphological measurement value concerning a reference phase of the plurality of phases and a morphological measurement value concerning a measurement target phase, whether the tumor has infiltrated the tubular tissue.

5. The apparatus of claim 1, wherein the image processing circuitry determine, based on a comparison between a change in morphological measurement value over the plurality of phases concerning the tubular tissue region and a change in morphological measurement value over the plurality of phases concerning a normal region, whether the tumor has infiltrated the tubular tissue.

6. The apparatus of claim 1, wherein the image processing circuitry set a measurement region in a luminal region of the tubular tissue region and measures the morphological measurement value of the measurement region.

7. The apparatus of claim 1, wherein the image processing circuitry set a plurality of measurement regions in a plurality of tubular tissue regions of a plurality of tubular tissues,
measure the morphological index value of each of the plurality of measurement regions, and
determine, based on a change in the morphological index value of each of the plurality of measurement regions over the plurality of phases, whether the tumor has infiltrated the tubular tissue.

8. The apparatus of claim 1, wherein the plurality of three-dimensional images are acquired by causing a medical image diagnostic apparatus to image, over the plurality of phases, an imaging region including an esophagus in which the tumor has arisen and the tubular tissue, and
the tubular tissue is one of a bronchus and a blood vessel near the esophagus.

9. The apparatus of claim 1, wherein the image processing circuitry measure, as the morphological index value, at least one of a luminal area, an inner circumference length, an outer circumference length, a long axis length, a short axis length, a ratio between the long axis length and the short axis length, and a degree of circularity of the tubular tissue region.

10. A medical image processing apparatus comprising image processing circuitry configured to process a plurality of three-dimensional images in a plurality of phases,
wherein the image processing circuitry set, with respect to the plurality of three-dimensional images in the plurality of phases, a first region of interest including a first peripheral portion region of a first peripheral portion with likelihood of tumor infiltration and a second region of interest including a second peripheral portion region of a second peripheral portion different from the first peripheral portion, and
determine, based on position variations of the first region of interest and the second region of interest over at least two phases of the plurality of phases, whether the tumor has infiltrated the first peripheral portion and the second peripheral portion.

11. The apparatus of claim 10, wherein the image processing circuitry set the second region of interest adjacent to the first region of interest.

12. The apparatus of claim 11, wherein the image processing circuitry set the first region of interest in a bronchial region existing around the tumor as the first peripheral portion region, and set the second region of interest in another anatomical region adjacent to the bronchial region as the second peripheral portion region.

13. The apparatus of claim 11, wherein the image processing circuitry measure a first position variation amount of the first region of interest and a second position variation amount of the second region of interest between at least two phases of the plurality of phases,
calculate a degree of similarity between the first position variation amount and the second position variation amount, and
determine, based on the degree of similarity, whether the tumor has infiltrated the first peripheral portion and the second peripheral portion.

14. A medical image processing apparatus comprising image processing circuitry configured to process a plurality of three-dimensional images in a plurality of phases,
wherein the image processing circuitry set, with respect to the plurality of three-dimensional images in the plurality of phases, a first region of interest including a peripheral portion region of a peripheral portion with likelihood of tumor infiltration and a second region of interest covering the first region of interest, and
determine, based on position variations of the first region of interest and the second region of interest over at least two phases of the plurality of phases, whether the tumor has infiltrated the peripheral portion.

15. The apparatus of claim 14, wherein the image processing circuitry further set a third region of interest including another peripheral portion region of another peripheral portion different from the peripheral portion, and
determine, based on position variations of the first region of interest, the second region of interest, and the third region of interest over the at least two phases, whether the tumor has infiltrated the peripheral portion.
